(19) United States
(12) Patent Application Publication
(10) Pub. No.: US 2010/0036498 A1
(43) Pub. Date: Feb. 11, 2010

(54) FUSION CAGE WITH REVERSE THREAD PROFILE (RTP)

(76) Inventor: Dennis McDevitt, Raleigh, NC (US)

Correspondence Address: Mark J. Pandiscio;Pandiscio & Pandiscio, P.C., 470 Totten Pond Road, Waltham, MA, 02451-1914, US

(21) Appl. No.: 12/418,058

(22) Filed: Apr. 3, 2009

Related U.S. Application Data
(60) Provisional application No. 61/072,829, filed on Apr. 3, 2008.

(51) Int. Cl. A61F 2/44; A61B 17/70

(52) U.S. Cl. 623/17.16; 606/246

(57) ABSTRACT

A bone fixation system for stabilizing two skeletal structures relative to one another, the system comprising:

a fusion cage comprising:
an elongated body having a distal end, a proximal end and a central axis extending therebetween; and
dovetail screw threads extending along the elongated body such that when the elongated body is disposed in the two skeletal structures, the dovetail screw threads inhibit movement of the skeletal structures relative to one another, even when the skeletal structures are subject to forces other than perpendicular compressive forces.

### REFERENCE TO PENDING PRIOR PATENT APPLICATION

This patent application claims benefit of pending prior U.S. Provisional Patent Application Ser. No. 61/072,829, filed Apr. 3, 2008 by Dennis McDevitt for FUSION CAGE WITH REVERSE THREAD PROFILE (RTP) (Attorney's Docket No. MCDEVITT-1 PROV), which patent application is hereby incorporated herein by reference.

### FIELD OF THE INVENTION

This invention relates to methods and apparatus for fusing together two adjacent bony structures in general and, more particularly, to methods and apparatus for fusing together two adjacent vertebral bodies.

### BACKGROUND OF THE INVENTION

The spinal column comprises a plurality of vertebral bodies separated by discs. These discs are essentially ligamentous pads or cushions disposed between adjacent vertebrae, and serve to accommodate various loads applied to the spinal column.

In some cases, a disc may undergo a painful deterioration due to injury, disease or other degenerative disorder. In some cases, the disc shrinks and flattens out, and the distance between the vertebral bodies begins to collapse. This can result in mechanical instability which can cause severe pain to the individual. In other cases, the side wall of the disc may weaken, resulting in a lateral bulging which can irritate sensitive adjacent anatomy, e.g., nerves. The pain associated with a deteriorating disc may be so severe that, in many cases, the disc must be removed and the adjacent vertebral bodies stabilized relative to one another. Typically, the adjacent vertebral bodies are stabilized by fusing the two bones together so that the adjacent vertebral bodies effectively become a single bony structure.

Successful spinal fusion generally requires sufficient bone ingrowth between the adjacent vertebral bodies to effectively create a singular solid bone mass. In this respect, it should be appreciated that the adjacent vertebral bodies need to be securely held in position relative to one another while such fusion ingrowth occurs since, when bone fusion is first initiated, the bone ingrowth is soft and lacks structural integrity. Accordingly, a variety of surgical devices have been developed to hold the adjacent vertebral bodies stationary relative to one another while bone fusion is effected.

In early spinal fusions, bone material was simply disposed between the adjacent vertebral bodies, typically at the posterior aspect of the vertebral bodies, and the spinal column was stabilized with a bone plate or rod spanning the adjacent vertebral bodies. However, the surgical procedures to implant the bone plate or rod were frequently relatively lengthy and involved. Furthermore, with this approach, once adequate bone fusion had been achieved, the hardware used to stabilize the adjacent vertebral bodies effectively became superfluous. However, this hardware was typically left in the body to avoid the necessity of a further surgical procedure.

In more recent fusion procedures, the disc is removed and the adjacent vertebral bodies are broadly fused across their respective end plates, without the use of anterior or posterior plating. More particularly, numerous devices have been developed for positioning in the intra-discal space, between the adjacent vertebral bodies, whereby to stabilize the adjacent vertebral bodies while bone fusion takes place.

These intra-discal fusion devices have taken many forms. One of the more successful designs (commonly referred to as a “fusion cage”) comprises a cylindrical (or modestly conical) implant having traditional screw threads (e.g., with an inverted V profile) along its exterior surface. These traditional screw threads facilitate insertion of the fusion cage into the intra-discal space and help stabilize the fusion cage relative to the adjacent vertebral bodies (and hence help stabilize the adjacent vertebral bodies relative to one another). The fusion cage is preferably hollow, with radial openings, so that the fusion cage can be filled with bone material to facilitate fusion of the adjacent vertebral bodies. Typically, two fusion cages are used, set in side-by-side relation.

More particularly, in spinal fusion procedures using conventional fusion cages, the damaged disc is first excised, the opposing end plates of the vertebral bodies are prepared, fusion cage seats are formed in the vertebral bodies (e.g., by drilling and tapping), and then the fusion cages are positioned between the adjacent vertebral bodies so as to support the vertebral bodies while bone fusion takes place.

While conventional fusion cages have proven to be a significant advance in the art, they also tend to suffer from at least one significant disadvantage. More particularly, while conventional fusion cages have proven to be capable of providing adequate stabilization for the vertebral bodies while those vertebral bodies are loaded with a perpendicular (relative to the vertebral end plates) compressive force (e.g., such as when the patient is sitting quietly), they frequently fail to provide adequate stabilization when the vertebral bodies are subjected to other forces. Even more particularly, conventional fusion cages have been found to provide inadequate stabilization when the vertebral bodies are subjected to oblique compressive forces and to lateral forces, and conventional fusion cages have been found to provide substantially no stabilization to the vertebral bodies when the vertebral bodies are subjected to tensile forces.

Thus, conventional fusion cages provide incomplete stabilization of the vertebral bodies relative to one another, and may permit some movement of the vertebral bodies to occur, which can inhibit proper fusion of the vertebral bodies.

In view of the foregoing, there is a need for an improved fusion cage which can help to ensure proper bone fusion even where the vertebral bodies are subjected to forces other than perpendicular compressive forces.

In addition to the foregoing, there is a need for an improved bone fixation system which can help to ensure proper bone fusion even where bone segments are subjected to forces other than perpendicular compressive forces.

### SUMMARY OF THE INVENTION

The present invention provides a novel system for fusing together two adjacent bony structures in general and, more particularly, the present invention provides a novel method and apparatus for fusing together two adjacent vertebral bodies.

The novel bone fixation system of the present invention comprises a novel fusion cage which is configured with a dovetail thread profile which can help to stabilize opposing vertebral bodies even when the vertebral bodies are subject to forces other than perpendicular compressive forces. In use, the surgeon first excises (in whole or in part) the damaged disc. Then the end plates of the vertebral bodies are prepared. Next, two fusion cage seats are formed in the vertebral bodies, e.g., by drilling and then tapping dovetail thread seats in the vertebral bodies. Then two novel fusion cages are installed, with the dovetail thread profiles engaging the dovetail thread seats, so that the fusion cages stabilize the opposing vertebral bodies relative to one another. Significantly, the undercut dovetail threads hold the adjacent vertebral bodies in position relative to one another even when the vertebral bodies are subjected to forces other than perpendicular compressive forces.

In another form of the present invention, the novel bone fixation system may be used to stabilize bony structures other than vertebral bodies, e.g., for fracture fixation to hold together bone segments, etc.

In another form of the present invention, there is provided a bone fixation system for stabilizing two skeletal structures relative to one another, the system comprising:

a fusion cage comprising:
an elongated body having a distal end, a proximal end and a central axis extending therebetween; and
dovetail screw threads extending along the elongated body such that when the elongated body is disposed in the two skeletal structures, the dovetail screw threads inhibit movement of the skeletal structures relative to one another, even when the skeletal structures are subject to forces other than perpendicular compressive forces.

In another form of the present invention, there is provided a method for stabilizing two skeletal structures relative to one another, the method comprising:

providing a fusion cage comprising:
an elongated body having a distal end, a proximal end and a central axis extending therebetween; and
dovetail screw threads extending along the elongated body such that when the elongated body is disposed in the two skeletal structures, the dovetail screw threads inhibit movement of the skeletal structures relative to one another, even when the skeletal structures are subject to forces other than perpendicular compressive forces;

forming dovetail seats in the two skeletal structures; and

positioning the fusion cage in the two skeletal structures such that the dovetail screw threads are disposed in the dovetail seats.

In another form of the present invention, there is provided a bone fixation system for stabilizing two skeletal structures relative to one another, the system comprising:

a fusion cage comprising:
an elongated body having a distal end, a proximal end and a central axis extending therebetween; and
constant pitch, tapered screw threads extending along the elongated body such that when the elongated body is disposed in the two skeletal structures, the constant pitch, tapered screw threads inhibit movement of the skeletal structures relative to one another.

In another form of the present invention, there is provided a method for stabilizing two skeletal structures relative to one another, the method comprising:

providing a fusion cage comprising:
an elongated body having a distal end, a proximal end and a central axis extending therebetween; and
constant pitch, tapered screw threads extending along the elongated body such that when the elongated body is disposed in the two skeletal structures, the constant pitch, tapered screw threads inhibit movement of the skeletal structures relative to one another, even when the skeletal structures are subject to forces other than perpendicular compressive forces;

forming thread seats in the two skeletal structures; and

positioning the fusion cage in the two skeletal structures so that the constant pitch, tapered screw threads are securely disposed in the thread seats.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 is a perspective view of a novel fusion cage formed in accordance with the present invention;

FIG. 2 is a side view, in section, of the novel fusion cage of FIG. 1;

FIGS. 3 and 4 are perspective views showing two of the novel fusion cages of FIG. 1 deployed between two vertebral bodies;

FIG. 5 is a side view, in section, showing the novel fusion cage of FIG. 1 deployed between two vertebral bodies;

FIG. 6 is a perspective view, in section, showing two of the novel fusion cages of FIG. 1 deployed between two vertebral bodies;

FIG. 7 is a side view of another novel fusion cage formed in accordance with the present invention;

FIG. 8 is a side view, in section, of the novel fusion cage of FIG. 7;

FIG. 9 is a side view of still another novel fusion cage formed in accordance with the present invention;

FIG. 10 is a side view of yet another novel fusion cage formed in accordance with the present invention;

FIG. 11 is a side view of still another novel fusion cage formed in accordance with the present invention;

FIG. 12 is a side view showing another novel fusion cage formed in accordance with the present invention;

FIG. 13 is a side view, in section, of the novel fusion cage shown in FIG. 12;

FIGS. 14 and 15 are perspective views showing novel fusion cages securing a prosthetic disc between two vertebral bodies; and

FIG. 16 is a side view showing novel fusion cages securing a prosthetic disc between two vertebral bodies.

### DETAILED DESCRIPTION OF THE PREFERRED EMBODIMENTS

The present invention provides a novel method and apparatus for fusing together two adjacent bony structures. For convenience, the present invention will hereinafter be discussed in the context of a spinal fusion procedure (e.g., for fusing together two opposing vertebral bodies). However, it should be appreciated that the present invention may also be used to stabilize other bony structures relative to one another while bone fusion takes place.

### Bone Fixation System With Solid Dovetail Threads

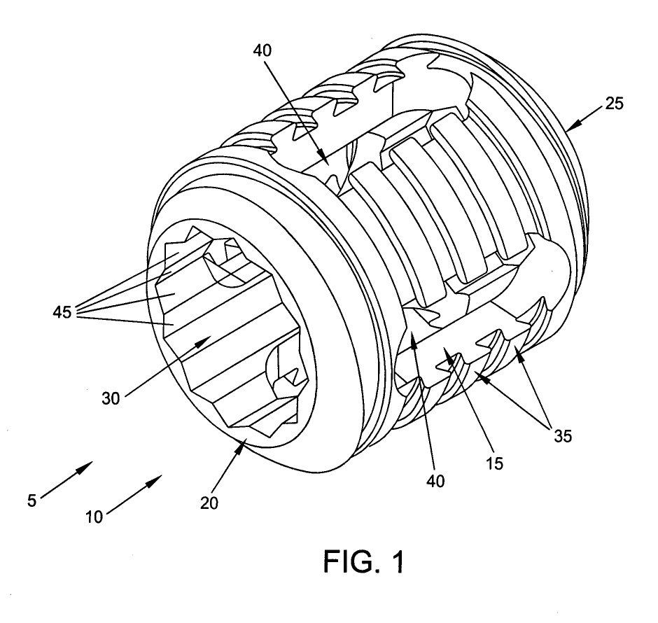

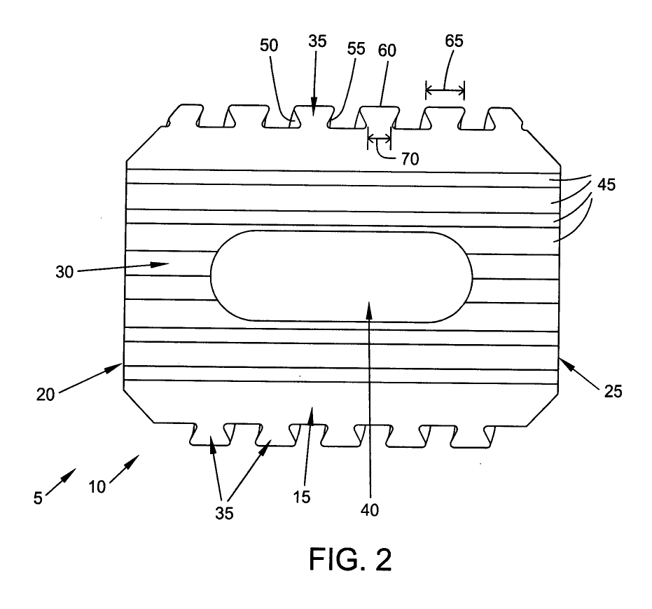

Looking first at FIGS. 1 and 2, there is shown a novel bone fixation system 5 formed in accordance with the present invention. Bone fixation system 5 generally comprises a novel fusion cage 10 for disposition between two bony structures (e.g., two vertebral bodies).

Fusion cage 10 generally comprises a body 15 having a distal end 20, a proximal end 25, and a central lumen 30 extending therebetween. Dovetail threads 35 are formed on the exterior surface of body 15. A plurality of windows 40 extend between the interior of central lumen 30 and the exterior of body 15.

Body 15 is configured to: (i) accommodate forces applied to adjacent vertebral bodies, and (ii) receive bone (or bone substitute) material for promoting fusion across the intra-discal space, as will hereinafter be discussed. Body 15 preferably has an elongated cylindrical (or modestly conical) shape so as to facilitate positioning in the intra-discal space.

Central lumen 30 extends between distal end 20 and proximal end 25. Central lumen 30 preferably accommodates an inserter, whereby to permit driving deployment of body 15 into the host bones, and central lumen 30 receives bone (or bone substitute) material, whereby to promote fusion across the intra-discal space. To this end, the side wall of central lumen 30 preferably has a non-circular profile so as to facilitate driving rotation by an inserter, as will hereinafter be discussed. By way of example, the side wall of central lumen 30 may be formed by a plurality of flat, non-parallel surfaces 45.

Dovetail threads 35 are configured to be rotationally received in the host bones. Dovetail threads 35 extend in a helical pattern about the outer surface of body 15. Dovetail threads 35 are preferably continuous, except for where they are interrupted by windows 40. Significantly, dovetail threads 35 have an undercut profile characterized by an inclined undercut leading surface 50, an inclined undercut trailing surface 55, and a flat peripheral surface 60, whereby to provide a solid thread profile having a larger periphery than interior. In other words, solid dovetail threads 35 have a peripheral dimension 65 which is longer than their base dimension 70. Thus, solid threads 35 have a reverse thread profile (RTP), in the sense that they are reversed from the traditional inverted V-shaped thread profile of conventional fusion cages. As a result, when fusion cage 10 is threadly engaged across adjacent vertebral bodies, dovetail threads 35 will interlock with the surrounding bone, whereby to inhibit movement of the vertebral bodies relative to one another. Significantly, on account of the dovetail profile of the screw threads, fusion cage 10 can hold the vertebral bodies in position relative to one another even when the vertebral bodies are subjected to forces other than perpendicular compressive forces. By way of example, the dovetail profile of the screw threads can hold the vertebral bodies in position even when the vertebral bodies are subjected to tensile forces.

Dovetail threads 35 may be formed integral with body 15 or, alternatively, they may be added to body 15 in ways well known in the art. In one preferred form of the invention, dovetail threads 35 are formed by machining away portions of body 15. In another preferred form of the invention, body 15 and dovetail threads 35 are formed as a single structure by molding.

Windows 40 communicate between the interior of central lumen 30 and the exterior of fusion cage 10. Windows 40 permit the bone (or bone substitute) material within the fusion cage to oseointegrate with the host bones, whereby to promote fusion across the intra-discal space. Body 15 preferably comprises four windows 40, however, body 15 may also comprise any other number of windows 40 consistent with the present invention (e.g., two, three, ten, etc.).

Fusion cage 10 is formed out of one or more biocompatible materials. These biocompatible materials may be non-absorbable (e.g., stainless steel, titanium, plastic or other suitable non-absorbable material), or absorbable (e.g., PLA), or osteoconductive or osteoinductive (e.g., ceramic, allograft or coral). In any case, fusion cage 10 is formed out of one or more materials having adequate strength characteristics consistent with the function of the fusion cage. It should be appreciated that it is not necessary for all of the components of fusion cage 10 to be formed out of the same material. In fact, a particular component may be formed out of a specific material or materials most advantageous for that particular component. Thus, different components may be formed out of different materials, different portions of a single component may be formed out of different materials, etc.

It is important that dovetail threads 35 of fusion cage 10 securely engage the bone of the adjacent vertebral bodies so as to lock the vertebral bodies against movement. To that end, it is preferred that good thread seats, having a dovetail profile matching that of the fusion cage threads, be prepared in the vertebral bodies which are to receive the dovetail threads 35. These dovetail thread seats may be formed in the host bone by providing self-tapping dovetail threads 35. More preferably, however, a separate tap is used to prepare appropriate dovetail thread seats in the vertebral bodies. This tap has a distal end which has a profile matching the profile of dovetail threads 35, so that when fusion cage 10 is inserted between the prepared vertebral bodies, dovetail threads 35 are snugly received by the corresponding dovetail thread seats formed in the vertebral bodies.

An inserter is used to deploy fusion cage 10. This inserter preferably drivingly mates with the side wall of central lumen 30, so that the inserter can rotationally advance fusion cage 10 into position.

Bone fixation system 5 is preferably used as follows.

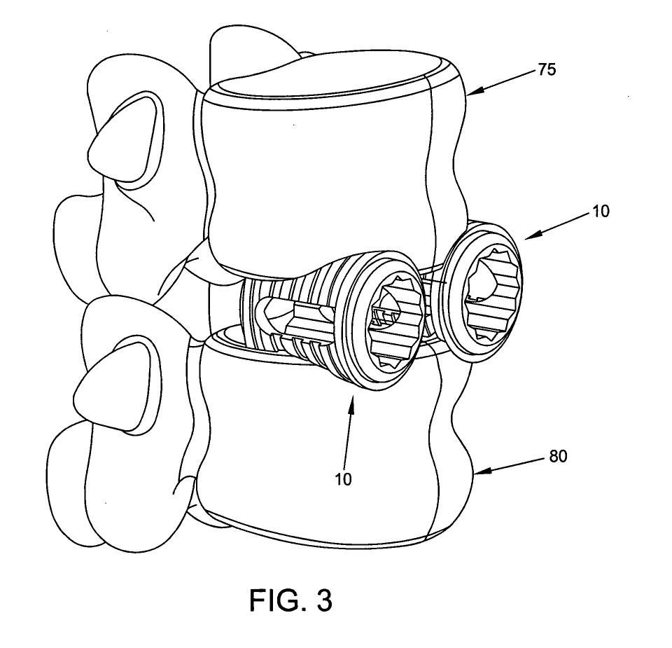

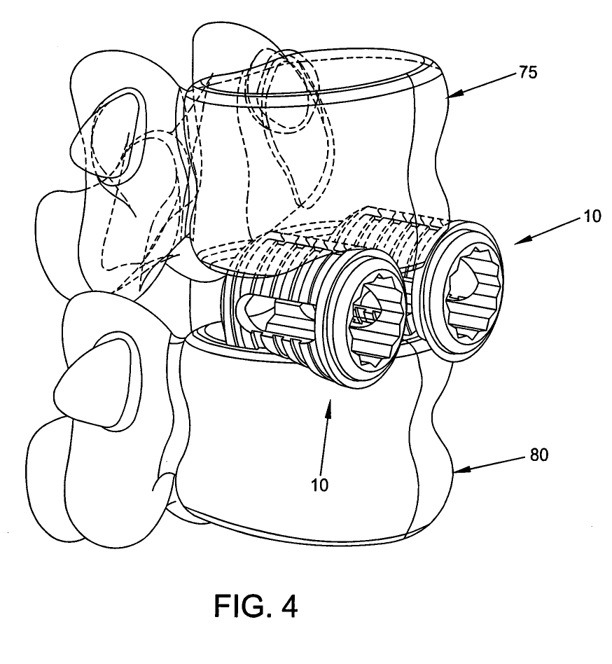

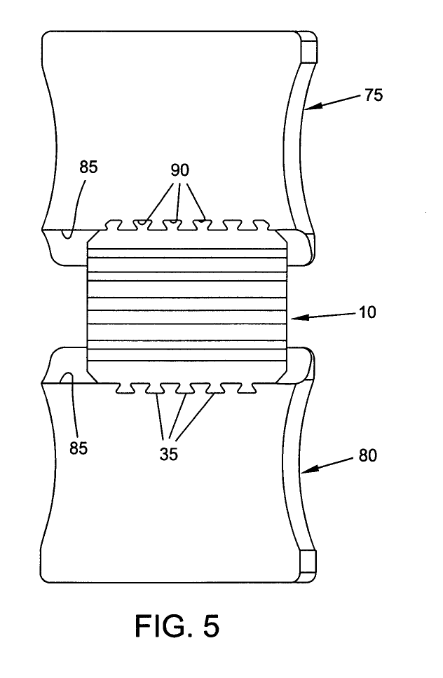

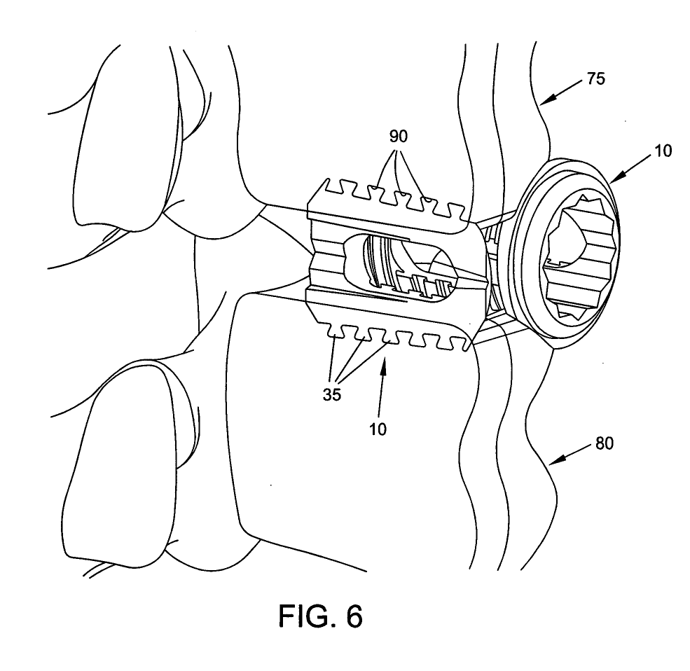

First, and looking now at FIGS. 3-6, the diseased or degenerative disc between two adjacent vertebral bodies 75 and 80 is removed (partially or completely) in ways well known in the art. Then recesses 85 (see FIG. 5) are drilled in the vertebral bodies in ways well known in the art. Next, the tap is used to form dovetail thread seats 90 (see FIG. 5) in the vertebral bodies.

Next, the inserter is positioned into central lumen 30 and used to rotationally advance a fusion cage 10 into position between the vertebral bodies. As this occurs, the body of fusion cage 10 is received within recess 85, with dovetail threads 35 being snugly received within dovetail thread seats 90. On account of the dovetail engagement between the screw threads 35 of the fusion cage and the dovetail thread seats 90 of the vertebral bodies, fusion cage 10 can hold the vertebral bodies in position relative to one another even when the vertebral bodies are subjected to forces other than perpendicular compressive forces. More specifically, while the vertebral bodies are subjected to perpendicular compressive forces, the bodies of the fusion cages serve to carry the load. However, when the vertebral bodies are subjected to oblique forces, and particularly to tensile forces, the dovetail profile of the screw threads holds the vertebral bodies in position relative to one another. This is a significant advance over conventional fusion cages.

Preferably two fusion cages are installed, in a side-by-side disposition, as shown in FIGS. 3, 4 and 6.

Finally, bone (or bone substitute) material is inserted into central lumen 30. By virtue of the windows 40, this bone (or bone substitute) material can oseointegrate with the bone masses of vertebral bodies 75 and 80, whereby to facilitate bone fusion.

### Bone Fixation System With Grooved Dovetail Threads

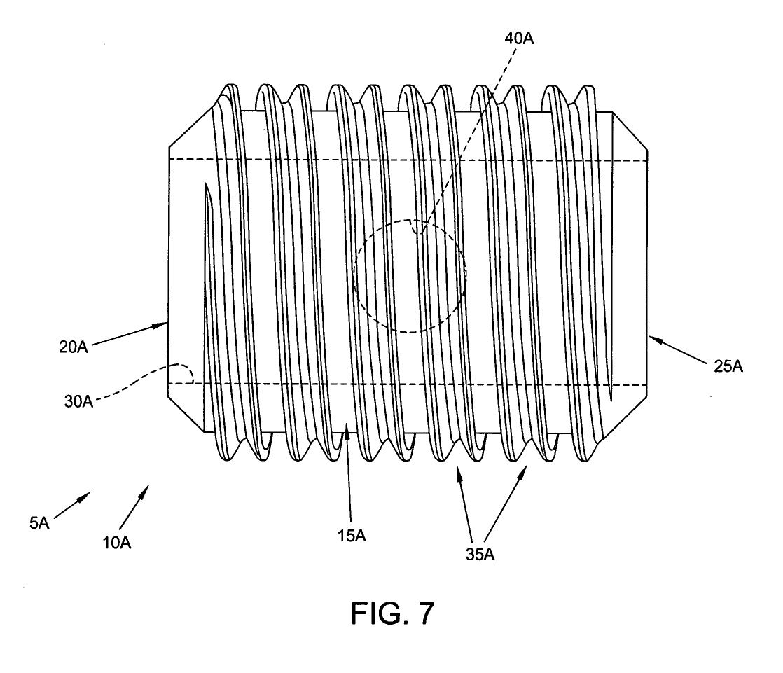

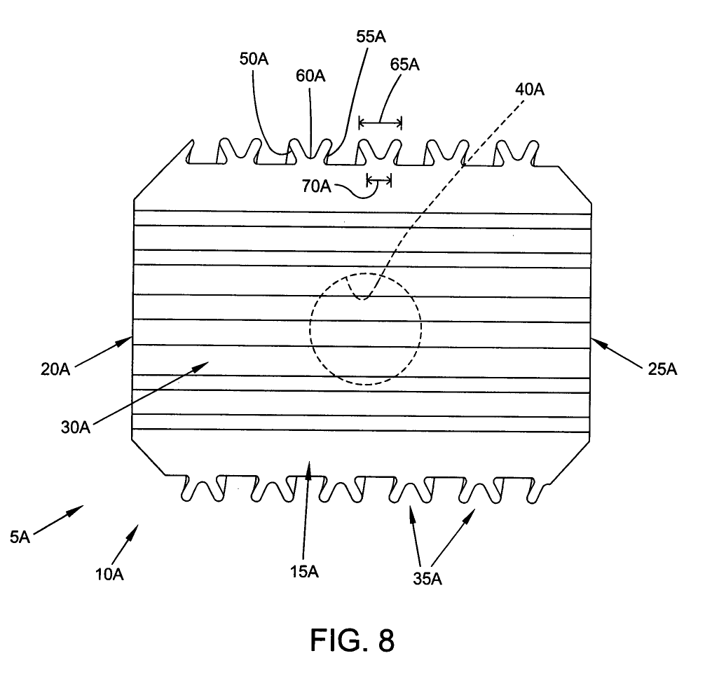

Looking now at FIGS. 7 and 8, there is shown an alternative bone fixation system 5A formed in accordance with the present invention. Bone fixation system 5A generally comprises a novel fusion cage 10A for disposition between two bony structures (e.g., two vertebral bodies).

Novel fusion cage 10A is substantially the same as fusion cage 10 discussed above, and is used in substantially the same manner as fusion cage 10 discussed above, except as will hereinafter be discussed. More particularly, fusion cage 10A generally comprises a body 15A having a distal end 20A, a proximal end 25A, and a central lumen 30A extending therebetween. Grooved dovetail threads 35A are formed on the exterior surface of body 15A. One or more windows 40A, extending between the interior of central lumen 30A and the exterior of body 15A, may be provided.

Grooved dovetail threads 35A are configured to be rotationally received in the host bones. Grooved dovetail threads 35A extend in a helical pattern about the outer surface of body 15A. Grooved dovetail threads 35A are preferably continuous, except for where they are interrupted by windows 40A. Significantly, grooved dovetail threads 35A have an undercut profile characterized by an inclined undercut leading surface 50A, an inclined undercut trailing surface 55A, and a grooved peripheral surface 60A, whereby to provide a thread profile having a larger periphery than interior. In other words, grooved dovetail threads 35A have a peripheral dimension 65A which is longer than the base dimension 70A. By providing dovetail threads 35A with a grooved peripheral surface 60A (as opposed to the solid peripheral surface 60 provided with the dovetail threads 35 of FIGS. 1 and 2), less of the host bone needs to be removed during tapping, and increased surface area contact is achieved between fusion cage 10A and the surrounding bone, whereby to further stabilize the elements relative to one another. When fusion cage 10A is threadly engaged across adjacent vertebral bodies, grooved dovetail threads 35A will interlock with the surrounding bone, whereby to inhibit movement of the vertebral bodies relative to one another. Significantly, on account of the dovetail profile of the screw threads, fusion cage 10A can hold the vertebral bodies in position relative to one another even when the vertebral bodies are subjected to forces other than perpendicular compressive forces. By way of example, the dovetail profile of the screw threads can hold the vertebral bodies in position even when the vertebral bodies are subjected to tensile forces.

### Bone Fixation System With Composite Dovetail Threads

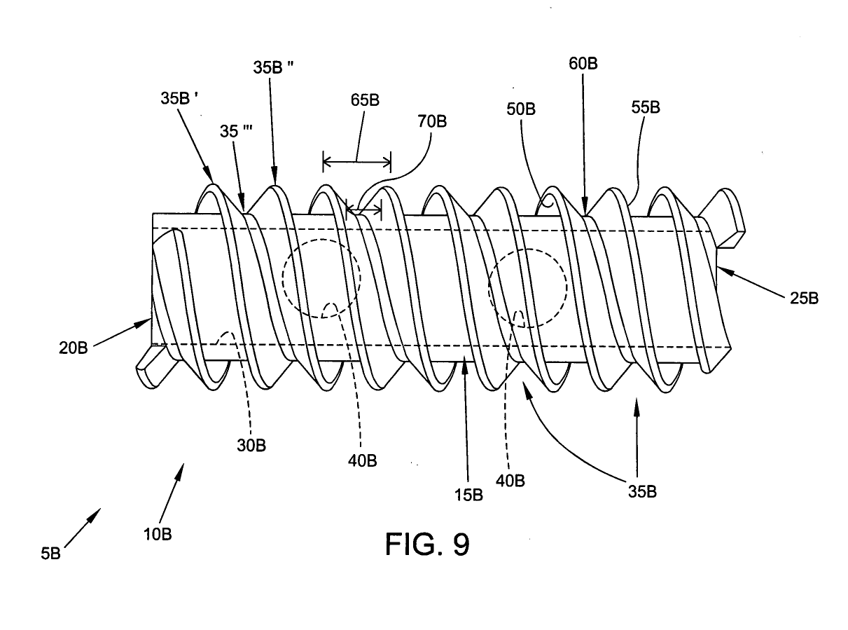

Looking now at FIG. 9, there is shown an alternative bone fixation system 5B formed in accordance with the present invention. Bone fixation system 5B generally comprises a novel fusion cage 10B for disposition between two bony structures (e.g., two vertebral bodies).

Novel fusion cage 10B is substantially the same as fusion cage 10 discussed above, and is used in substantially the same manner as fusion cage 10 discussed above, except as will hereinafter be discussed. More particularly, fusion cage 10B generally comprises a body 15B having a distal end 20B, a proximal end 25B, and a central lumen 30B extending therebetween. Composite grooved dovetail threads 35B are formed on the exterior surface of body 15B. One or more windows 40B, extending between the interior of central lumen 30B and the exterior of body 15B, may be provided.

Composite grooved dovetail threads 35B are configured to be rotationally received in the host bones. Composite grooved dovetail threads 35B extend in a helical pattern about the outer surface of body 15B. Composite grooved dovetail threads 35B are preferably continuous, except for where they are interrupted by windows 40B. Significantly, composite grooved dovetail threads 35B have an undercut profile characterized by an inclined undercut leading surface 50B, an inclined undercut trailing surface 55B, and a grooved peripheral surface 60B, whereby to provide a thread profile having a larger periphery than interior. In other words, composite grooved dovetail threads 35B have a peripheral dimension 65B which is longer than the base dimension 70B. Significantly, in this form of the invention, the composite grooved dovetail threads 35B can actually be formed by two separate angled undercut threads 35B′ and 35B″, with the gap 35B″′ (located between the threads 35B′ and 35B″) forming the grooved peripheral surface 60B. Again, by providing dovetail threads 35B with a grooved peripheral surface 60B (as opposed to the solid peripheral surface 60 provided with the dovetail threads 35 of FIGS. 1 and 2), less of the host bone needs to be removed during tapping, and increased surface area contact is achieved between fusion cage 10B and the surrounding bone, whereby to further stabilize the elements relative to one another. When fusion cage 10B is threadly engaged across adjacent vertebral bodies, composite grooved dovetail threads 35B will interlock with the surrounding bone, whereby to inhibit movement of the vertebral bodies relative to one another. Significantly, on account of the dovetail profile of the screw threads, fusion cage 10B can hold the vertebral bodies in position relative to one another even when the vertebral bodies are subjected to forces other than perpendicular compressive forces. By way of example, the dovetail profile of the screw threads can hold the vertebral bodies in position even when the vertebral bodies are subjected to tensile forces.

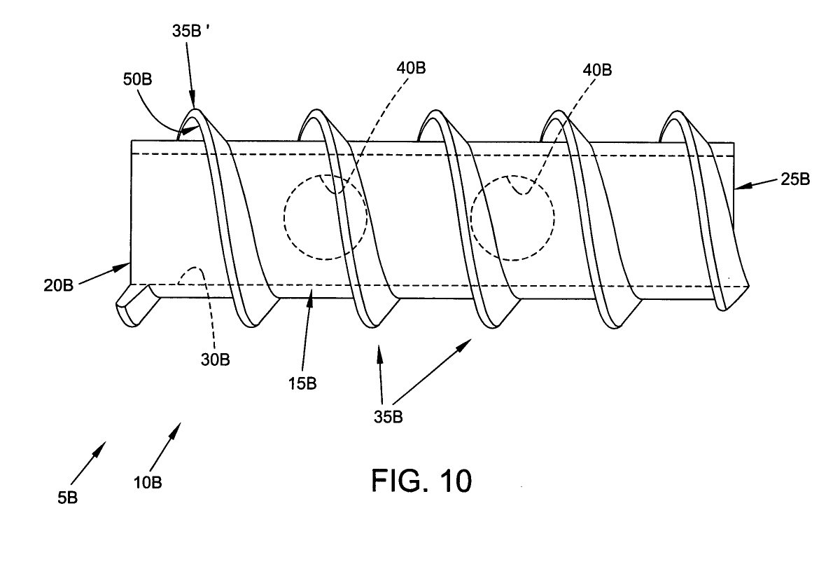

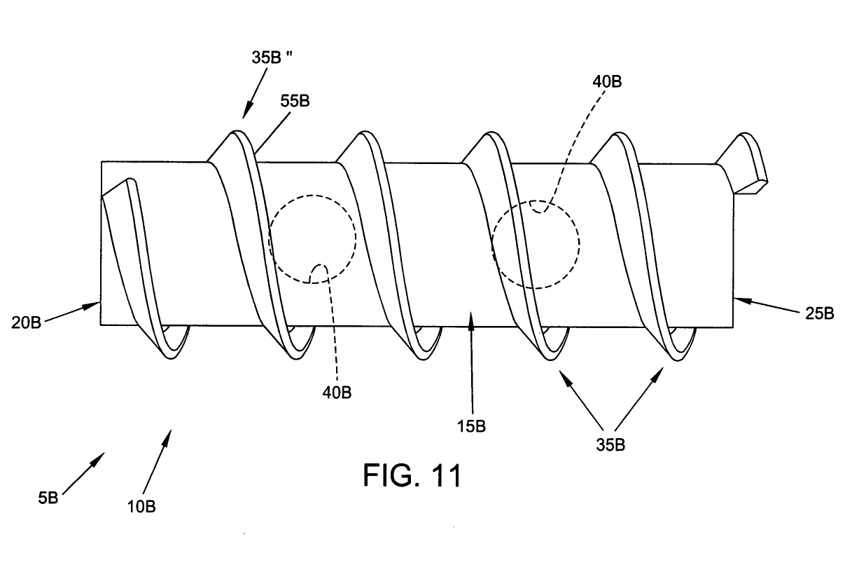

If desired, one of the separate angled threads 35B′ or 35B″ may be omitted, whereby to provide partial dovetail threads 35B. Thus, for example, in FIG. 10, the angled threads 35B′ (comprising inclined leading surface 50B) are provided, and the angled threads 35B″ are omitted. Alternatively, as can be seen in FIG. 11, the angled threads 35B″ (comprising inclined trailing surface 55B) are provided, and the angled threads 35B′ are omitted.

### Bone Fixation System With Tapering Threads

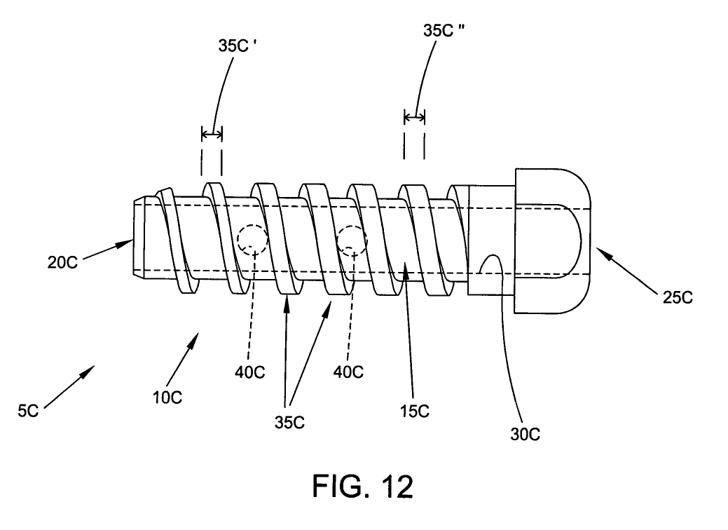

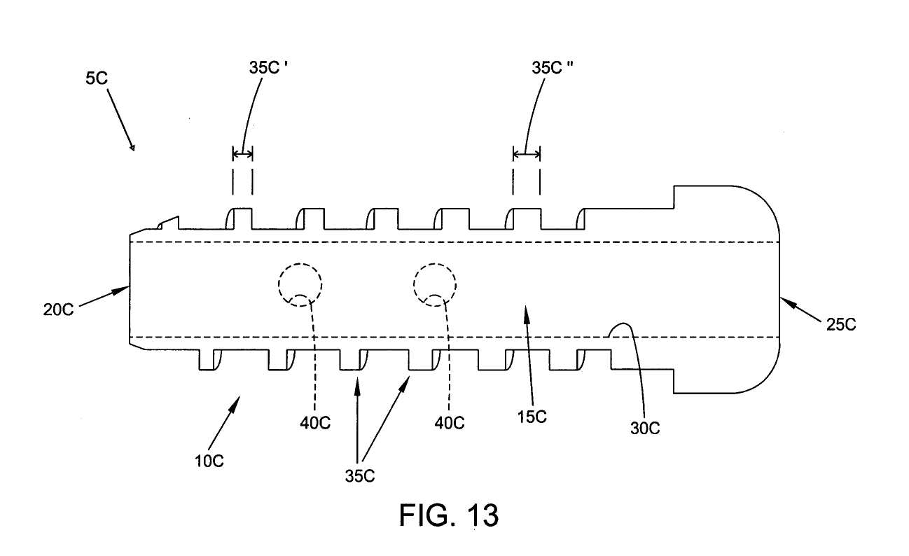

Looking now at FIGS. 12 and 13, there is shown an alternative bone fixation system 5C formed in accordance with the present invention. Bone fixation system 5C generally comprises a novel fusion cage 10C for disposition between two bony structures (e.g., two vertebral bodies).

Novel fusion cage 10C is substantially the same as fusion cage 10 discussed above, and is used in substantially the same manner as fusion cage 10 discussed above, except as will hereinafter be discussed. More particularly, fusion cage 10C generally comprises a body 15C having a distal end 20C, a proximal end 25C, and a central lumen 30C extending therebetween. Constant pitch, tapered threads 35C are formed on the exterior surface of body 15C. One or more windows 40C, extending between the interior of central lumen 30C and the exterior of body 15C, may be provided.

Constant pitch, tapered threads 35C are configured to be rotationally received in the host bones. Constant pitch, tapered threads 35C extend in a helical pattern about the outer surface of body 15C. Constant pitch, tapered threads 35C are preferably continuous, except for where they are interrupted by windows 40C. Significantly, constant pitch, tapered threads 35C have a tapered thread form which increases in the distal-to-proximal direction, while maintaining a constant pitch. In other words, the thickness of the tapered thread 35C′ is greater than the thickness of the tapered thread 35C″. As a result of this construction, when thread seats are tapped in the vertebral bodies which have a recess sized to receive the tapered thread 35C′, the thicker trailing threads (e.g., 35C″) will make a snug fit in the tapped thread seats, thereby helping stabilize the fusion cage in bone.

If desired, constant pitch, tapered threads 35C may have a rectangular cross-section (e.g., such as is shown in FIGS. 12 and 13) or, more preferably, tapered threads 35C may have a dovetail cross-section (e.g., such as the various dovetail constructions shown in FIGS. 1-11).

### Use With Prosthetic Discs And/Or Other Bony Structures

In the foregoing discussion of the novel bone fixation systems 5, 5A, 5B and 5C, the fusion cages 10, 10A, 10B and 10C are discussed in the context of fusing together two vertebral bodies. However, it should also be appreciated that fusion cages 10, 10A, 10B and 10C may be used in other applications.

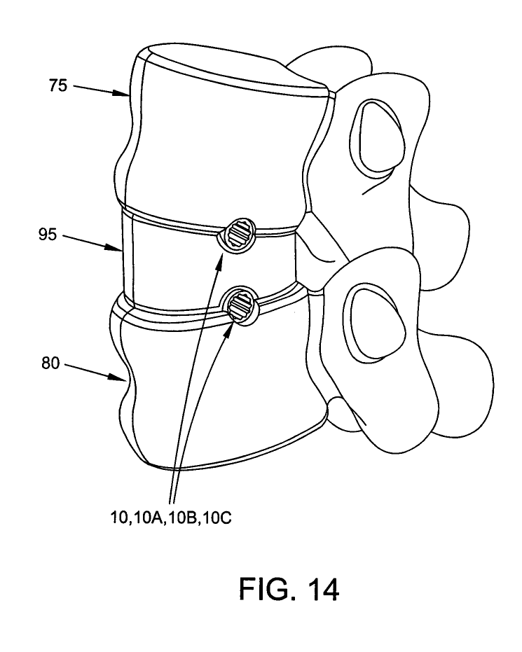

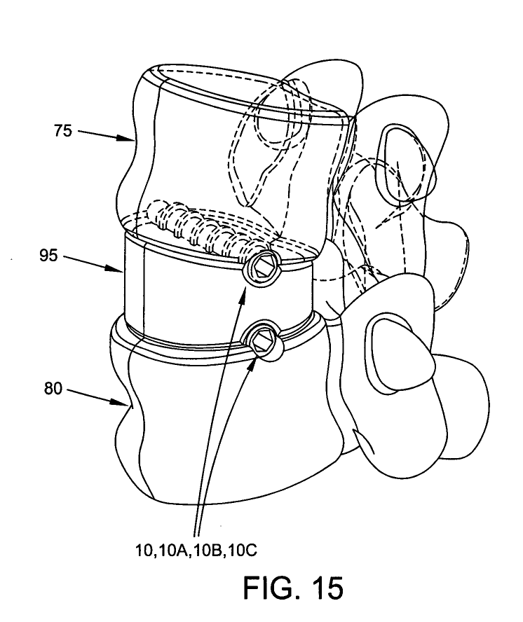

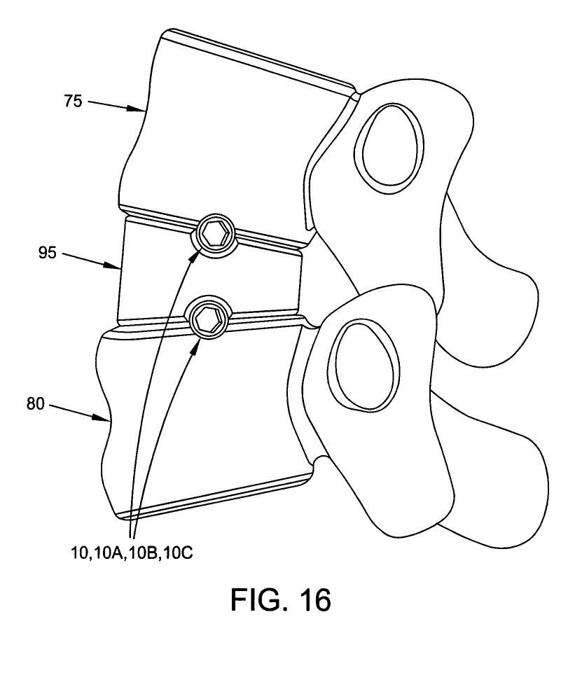

Thus, for example, and looking now at FIGS. 12-14, fusion cages 10, 10A, 10B and 10C are shown securing a prosthetic disc 95 in position between vertebral bodies 75 and 80.

Furthermore, fusion cages 10, 10A, 10B and 10C can be used for a wide range of other bone fixation applications, e.g., fracture fixation. In such other applications, one or more features may be omitted, depending on the application involved. Thus, for example, in a so called “long bone” fixation, central lumen 30 (30A, 30B, 30C) and/or windows 40 (40A, 40B, 40C) may be omitted.

### Further Modifications

It will be understood that many additional changes in the details, materials, steps and arrangements of parts, which have been herein described and illustrated in order to explain the nature of the invention, may be made by those skilled in the art while remaining within the principles and scope of the present invention.

What is claimed is:

1. A bone fixation system for stabilizing two skeletal structures relative to one another, the system comprising:
a fusion cage comprising:
an elongated body having a distal end, a proximal end and a central axis extending therebetween; and
dovetail screw threads extending along the elongated body such that when the elongated body is disposed in the two skeletal structures, the dovetail screw threads inhibit movement of the skeletal structures relative to one another, even when the skeletal structures are subject to forces other than perpendicular compressive forces.

2. A system according to claim 1 wherein the skeletal structures comprise at least one vertebral body.

3. A system according to claim 1 wherein the skeletal structures comprise at least one prosthetic disc.

4. A system according to claim 1 wherein the elongated body comprises a central lumen extending from the distal end to the proximal end.

5. A system according to claim 4 wherein the central lumen has a non-circular cross-section.

6. A system according to claim 4 wherein the elongated body further comprises at least one window extending transversely through the body and communicating with the central lumen.

7. A system according to claim 1 wherein the dovetail threads comprise solid dovetail threads.

8. A system according to claim 1 wherein the dovetail threads comprise grooved dovetail threads.

9. A system according to claim 1 wherein the dovetail threads comprise composite dovetail threads.

10. A system according to claim 9 wherein the dovetail threads comprise angled undercut threads.

11. A system according to claim 1 wherein the dovetail threads comprise a constant pitch, tapering dovetail thread.

12. A method for stabilizing two skeletal structures relative to one another, the method comprising:
providing a fusion cage comprising:
an elongated body having a distal end, a proximal end and a central axis extending therebetween; and
dovetail screw threads extending along the elongated body such that when the elongated body is disposed in the two skeletal structures, the dovetail screw threads inhibit movement of the skeletal structures relative to one another, even when the skeletal structures are subject to forces other than perpendicular compressive forces;
forming dovetail seats in the two skeletal structures; and
positioning the fusion cage in the two skeletal structures such that the dovetail screw threads are disposed in the dovetail seats.

13. A method according to claim 12 wherein the elongated body comprises a central lumen extending from the distal end to the proximal end, and at least one window extending transversely through the body and communicating with the central lumen, and further wherein the central lumen and window are filled with bone or bone substitute material after the fusion cage is positioned within the two skeletal structures.

14. A bone fixation system for stabilizing two skeletal structures relative to one another, the system comprising:
a fusion cage comprising:
an elongated body having a distal end, a proximal end and a central axis extending therebetween; and
constant pitch, tapered screw threads extending along the elongated body such that when the elongated body is disposed in the two skeletal structures, the constant pitch, tapered screw threads inhibit movement of the skeletal structures relative to one another.

15. A system according to claim 14 wherein the skeletal structures comprise at least one vertebral body.

16. A system according to claim 14 wherein the skeletal structures comprise at least one prosthetic disc.

17. A system according to claim 14 wherein the elongated body comprises a central lumen extending from the distal end to the proximal end.

18. A system according to claim 17 wherein the central lumen has a non-circular cross-section.

19. A system according to claim 17 wherein the elongated body further comprises at least one window extending transversely through the body and communicating with the central lumen.

20. A system according to claim 14 wherein the constant pitch, tapered threads comprise dovetail threads.

21. A system according to claim 20 wherein the constant pitch, tapered threads comprise solid dovetail threads.

22. A system according to claim 20 wherein the constant pitch, tapered threads comprise grooved dovetail threads.

23. A system according to claim 20 wherein the constant pitch, tapered threads comprise composite dovetail threads.

24. A system according to claim 23 wherein the constant pitch, tapered threads comprise angled undercut threads.

25. A method for stabilizing two skeletal structures relative to one another, the method comprising:
providing a fusion cage comprising:
an elongated body having a distal end, a proximal end and a central axis extending therebetween; and
constant pitch, tapered screw threads extending along the elongated body such that when the elongated body is disposed in the two skeletal structures, the constant pitch, tapered screw threads inhibit movement of the skeletal structures relative to one another, even when the skeletal structures are subject to forces other than perpendicular compressive forces;
forming thread seats in the two skeletal structures; and
positioning the fusion cage in the two skeletal structures so that the constant pitch, tapered screw threads are securely disposed in the thread seats.

26. A method according to claim 25 wherein the elongated body comprises a central lumen extending from the distal end to the proximal end, and at least one window extending transversely through the body and communicating with the central lumen, and further wherein the central lumen and window are filled with bone or bone substitute material after the fusion cage is positioned within the two skeletal structures.
